(19) United States
(12) Patent Application Publication
(10) Pub. No.: US 2010/0114273 A1
(43) Pub. Date: May 6, 2010

(54) ELECTRODE FOR FUNCTIONAL ELECTRICAL STIMULATION

(76) Inventor: Philip Edward Muccio, Ypsilanti, MI (US)

Correspondence Address: JELIC PATENT SERVICES, LLC, 2922 MARSHALL ST, ANN ARBOR, MI, 48108, US

(21) Appl. No.: 12/608,957

(22) Filed: Oct. 29, 2009

Related U.S. Application Data
(60) Provisional application No. 61/109,809, filed on Oct. 30, 2008.

(51) Int. Cl. A61N 1/04

(52) U.S. Cl. 607/115

(57) ABSTRACT

The present disclosure provides a hydrogel electrode designed to be integrated within a wearable functional electrode stimulation (FES) system. The wearable FES system comprises a portable electrical stimulator; wearable and stretchable clothing; an electrical connection between the electrical stimulator and the wearable and stretchable clothing; means to carry a current within the clothing; and one or more hydrogel electrode(s) which interface between the clothing and an applicable body part.

### CROSS-REFERENCE TO RELATED APPLICATIONS

This application claims the benefit of U.S. Provisional Application 61/109,809 filed Oct. 30, 2008. The content of this prior application is incorporated by reference in its entirety.

### BACKGROUND OF THE INVENTION

Functional Electrical Stimulation (FES) is a method of applying one or more electrical impulses to the body. FES may provide the benefits of pain management, muscle building, prevention of muscle atrophy, and muscle re-education of residual limb and/or peri-residual limb muscles.

### BRIEF SUMMARY OF THE INVENTION

The present disclosure provides a hydrogel electrode designed to be integrated within a wearable FES system. The wearable FES system comprises a portable electrical stimulator; wearable and stretchable clothing; an electrical connection between the electrical stimulator and clothing; means to carry a current within the clothing; and a plurality of hydrogel electrodes which interface between the clothing and an applicable body part.

The hydrogel electrode is made using polyethylene glycol (PEG) polymer hydrogel. In one embodiment, PEG hydrogels are created through controlled photo-initiated crosslinking of PEG-diacrylate polymers in the presence of water. This process produces a polymer network which is hydrophilic, highly absorbent (water up to 99% volume), and insoluble.

In one embodiment, a pressure sensitive adhesive (PSA) is bonded to one side of the hydrogel electrode. The PSA consists primarily of acrylates which are covalently bonded to the hydrogel electrode with photo-initiated crosslinking The side of the hydrogel electrode without the PSA is free to interact with the skin of the applicable body part.

### BRIEF DESCRIPTION OF THE DRAWINGS

FIG. 1 shows a comparison between a PEG polymer hydrogel electrode which is directly attached to the fabric versus an attachment to another adhesive which then attaches to the fabric.

FIG. 2 shows 8 millimeter diameter boluses of PEG hydrogel.

FIG. 3 shows thin film hydrogel samples.

FIG. 4 shows gold nanoparticle doped hydrogels.

FIG. 5 shows a wearable FES system with integrated hydrogel electrodes.

### DETAILED DESCRIPTION OF THE INVENTION

Wearable FES systems have been created to provide one or more electrical impulses to the body. Current FES systems typically use one or more electrodes consisting of either a conductive gel, conductive polymer, or a conductive polymer blend.

The conductive gel is the type which is typically used for ultrasound applications. The gel is contained in a wearable pouch and is transferred to the site of contact (between the FES system and the body) by diffusion through porous fabric. The gel is sticky although generally biocompatible. This method has the disadvantage of requiring continuous body contact with the gel, requiring periodic replenishment of the gel, and of not being suitable for weight-bearing locations.

The conductive polymer and conductive polymer blend electrodes have adhesive on both sides. One side of the electrode is attached to the body using adhesive, while the other side is attached to the FES system. This method is suitable for weight bearing applications and does not require exposure to sticky fluids. However, the method creates skin irritation in some people, may have high profile limiting application in some areas (e.g. feet), and can detach from the intended location when used chronically.

The present disclosure describes a novel PEG polymer hydrogel electrode for wearable FES systems which doesn't have the disadvantages of the conductive gel, conductive polymer, or conductive polymer blend electrodes. Specifically, the PEG polymer hydrogel electrode is designed to be conductive; provide a conformal interaction with the body; not induce skin irritation; secure snugly to fabric; not adhere strongly to skin; possess a low profile; have stretch characteristics in applications that require the electrode to stretch; and have properties which provide the wearer with at least 18 hours of use without drying.

The PEG polymer hydrogel electrode is designed to be conductive since it is hydrophilic and can be up to 99% water by volume. Although pure water isn't conductive, any salts in the water will enable conductivity.

The PEG polymer hydrogel electrode is designed to provide a conformal interaction with the body. This is because the PEG polymer hydrogels may be synthesized with a range of stiffness and water contents, which can alter the elasticity of the PEG hydrogel.

One unexpected result is that the PEG polymer hydrogel may be useful in situations where body hair is present. This would eliminate the step of shaving prior to applying the PEG polymer hydrogel electrode.

The PEG polymer hydrogel electrode is designed to not induce skin irritation. This is because PEG is highly biocompatible and is used in a number of medical and consumer products.

The PEG polymer hydrogel electrode is designed to secure snugly to fabric. In one embodiment, an adhesive would be covalently bonded to the fabric side of the hydrogel. This eliminates delamination concerns. The hydrogel with covalently bonded adhesive may be directly attached to the fabric or another adhesive, which is then attached to the fabric.

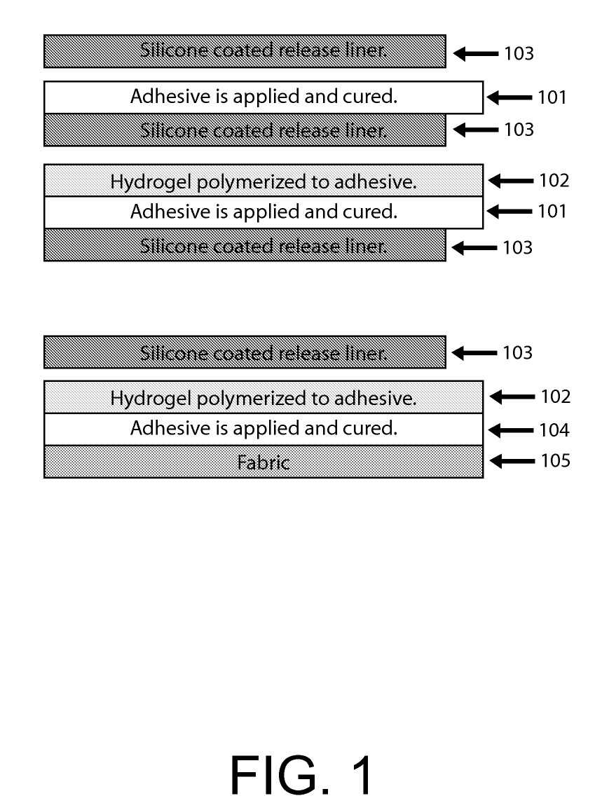

FIG. 1 shows a comparison between a PEG polymer hydrogel electrode which is directly attached to the fabric versus an attachment to another adhesive which then attaches to the fabric. Adhesive 101 is applied to the PEG polymer hydrogel electrode 102 and cured. A liner 103 is removed from the adhesive 101. A second adhesive 104 may be applied before attachment to the fabric 105.

The PEG polymer hydrogel electrode is designed to not adhere strongly to skin. PEPG hydrogels are known for their ability to resist cell adhesion and protein adsorption. They are not tacky so they don't adhere too easily to the skin.

The PEG polymer hydrogel electrode is designed to possess a low profile. This enables possible application at the foot. Hydrogels can be synthesized in a range of shapes and sizes through molding. Film thickness can be controlled by spin coating or drop casting. Films may be created as thin as roughly 500 microns.

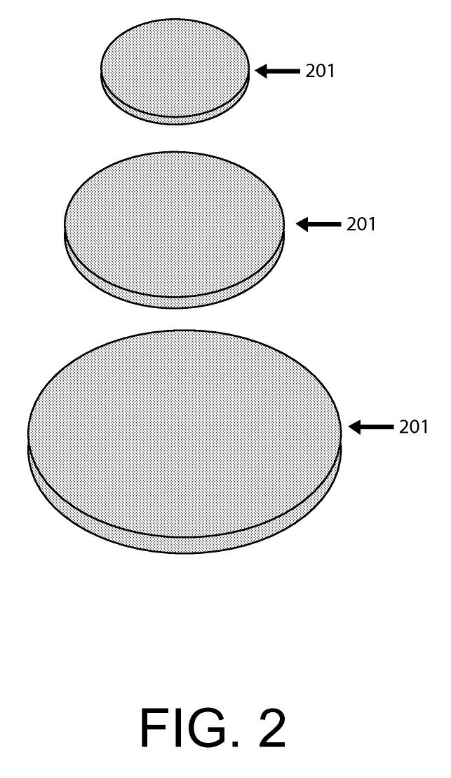

FIG. 2 shows 8 millimeter diameter boluses of PEG hydrogel. The boluses 201 may be molded to a variety of shapes.

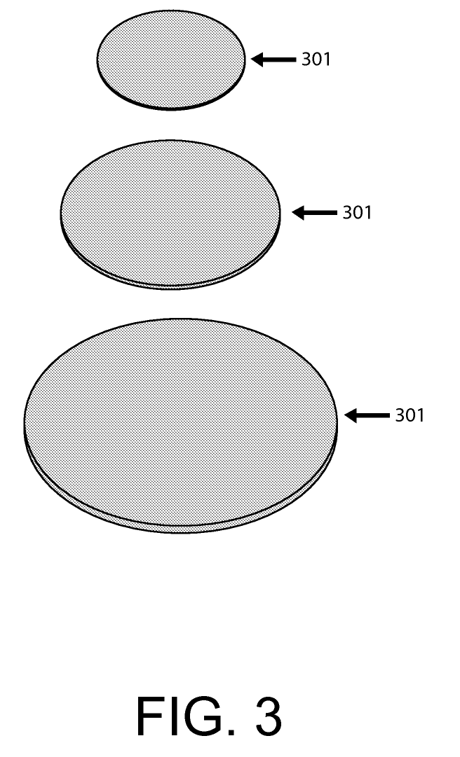

FIG. 3 shows thin film hydrogel samples. The thin film hydrogel samples 301 are roughly 500 microns thick.

The PEG polymer hydrogel electrode is designed to have stretch characteristics in applications that require the electrode to stretch. One example of a possible application is on the foot. While hydrogels are compressive, their crosslinks limit their ability to stretch. Typically, only 10% elongation may be achieved with hydrogel material. However, doping the hydrogel with a material which has elongation properties (e.g. polyurethane) may add elongation capability to the PEG polymer hydrogel electrode.

The PEG polymer hydrogel electrode is designed to have properties which provide the wearer with at least 18 hours of use without drying. In fact, the PEG polymer hydrogel electrode is expected to last weeks or months since it can remain hydrated that long.

In some patients, the hydrogel may cause skin irritation. In one embodiment, the hydrogel may be doped with a skin conditioner (e.g. aloe vera or vitamin C).

Many embodiments of the PEG polymer hydrogel electrode are possible. For example by varying the size the polyethylene glycol molecules and/or the crosslinking procedure, a wide variation in molecular weights (MW) is possible. A 400 MW hydrogel is expected to be stiffer than a 4000 MW hydrogel.

Another embodiment of the PEG polymer hydrogel electrode uses conductive nanoparticles such as gold or silver to increase conductivity of the PEG polymer hydrogel electrode. The nanoparticles may be entrained in the hydrogel matrices indefinitely, provided that they are larger than the hydrogel pore size.

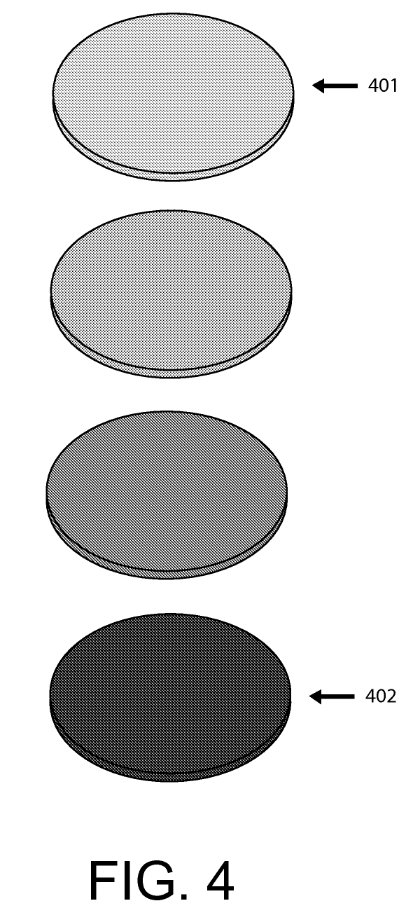

FIG. 4 shows gold nanoparticle doped hydrogels. Hydrogel with a low concentration of gold 401 has a lighter shade while hydrogel with high concentration of gold 402 has a darker shade.

The hydrogel electrode is made using polyethylene glycol (PEG) polymer hydrogel. In one embodiment, PEG hydrogels are created through controlled photo-initiated crosslinking of PEG-diacrylate polymers in the presence of water. This process produces a polymer network which is hydrophilic, highly absorbent (water up to 99% volume), and insoluble.

In one embodiment, a pressure sensitive adhesive (PSA) is bonded to one side of the hydrogel electrode. The PSA consists primarily of acrylates which are covalently bonded to the hydrogel electrode with photo-initiated crosslinking The side of the hydrogel electrode without the PSA is free to interact with the skin of the applicable body part.

The wearable FES system comprises a portable electrical stimulator; wearable and stretchable clothing; an electrical connection between the electrical stimulator and clothing; means to carry a current within the clothing; a plurality of thin planar conductive electrodes; and a plurality of hydrogel electrodes which interface between the thin planar conductive electrodes and an applicable body part.

The portable electrical stimulator is typically battery operated.

The wearable and stretchable clothing uses a spandex-like material. The spandex-like material provides stretchability and flexibility.

The electrical connection between the electrical simulator and clothing comprises two wires. One wire carries a positive direct current while the other wire carries a negative direct current.

Conductive elastic fabric conductors comprising silver provide the means to carry a current within the clothing.

The hydrogel electrode is comprised of a PEG polymer hydrogel, as described earlier in this disclosure.

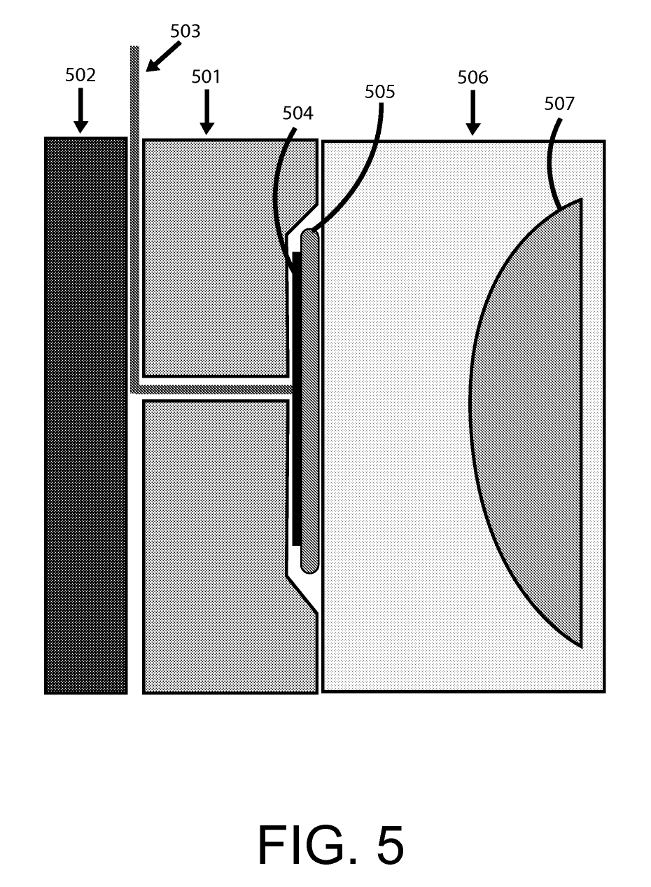

FIG. 5 shows a wearable FES system with integrated hydrogel electrodes. A portable electrical stimulator 501 is used to provide a direct current. The direct current is carried via a pair of wires 502 to an article of wearable and stretchable clothing 503. The wearable and stretchable clothing 503 contains conductive elastic fabric conductors 504 which carry the current to thin planar conductive electrodes comprised of silver fabric 505. The silver fabric electrodes carry the current to hydrogel electrodes 506.

While the present invention has been described herein with reference to an embodiment and various alternatives thereto, it should be apparent that the invention is not limited to such embodiments. Rather, many variations would be apparent to persons of skill in the art without departing from the scope and spirit of the invention, as defined herein and in the claims.

I claim:

1. A process for electrification of a plurality of hydrogel electrodes comprising:
using a portable electrical stimulator to provide a current;
carrying the current with wires, one for each electrode, to an article of clothing;
using conductive elastic fabric conductors within the clothing to carry the current to thin planar conductive fabric electrodes; and
using the thin planar conductive fabric electrodes to carry the current to the hydrogel electrodes.

2. The process of claim 1, wherein the conductive fabric comprises silver.

3. The process of claim 2, wherein the article of clothing is wearable by a mammal and stretchable.

4. The process of claim 3, wherein the hydrogel electrodes are applied to the skin of a mammal.

5. The process of claim 4, wherein the hydrogel electrodes are composed of polyethylene glycol polymer.

6. The process of claim 5, wherein the hydrogel electrodes are doped with conductive nanoparticles.

7. An improved wearable functional electrical stimulation system having a portable electrical stimulator to provide direct current, a pair of wires to carry the current to an article of clothing, means to carry the current within the clothing, and one or more electrodes to transfer current from the clothing to the body of a mammal, wherein the improvement comprises:
electrodes composed of polyethylene glycol polymer hydrogel.

8. The improved system of claim 7, wherein the electrodes composed of polyethylene glycol polymer hydrogel are doped with conductive nanoparticles.
